(19) United States
(12) Patent Application Publication
(10) Pub. No.: US 2022/0202340 A1
(43) Pub. Date: Jun. 30, 2022

(54) System, Method, and Apparatus for Visualizing Cardiac Activation

(71) Applicant: St. Jude Medical, Cardiology Division, Inc., St. Paul, MN (US)

(72) Inventors: Mark Hagfors, North Oaks, MN (US); Simon Pranaitis, Sammamish, WA (US); Don Curtis Deno, Andover, MN (US); Emma Davis, Saint Paul, MN (US)

(21) Appl. No.: 17/606,112

(22) PCT Filed: Apr. 22, 2020
(86) PCT No.: PCT/US2020/029265

§ 371 Date: Oct. 25, 2021

Related U.S. Application Data
(60) Provisional application No. 62/838,013, filed on Apr. 24, 2019.

(51) Int. Cl. A61B 5/341; A61B 5/339; A61B 5/287; G09B 23/28

(52) U.S. Cl. CPC A61B 5/341; G09B 23/28; A61B 5/287; A61B 5/339

(57) ABSTRACT

An animated cardiac activation map can be created by simulating particle flow over a three-dimensional representation of a cardiac surface. In particular, an electroanatomical mapping system can simulate and display particle flow through a conduction velocity map for the cardiac surface, with the conduction velocity map defining a conduction velocity vector field over the cardiac surface. Particles may be spawned randomly and/or according to a local activation timing map for the cardiac surface. Likewise, particle simulation may be displayed for a preset time interval and/or for a time interval determined by the local activation timing map. Particle simulation may also end if a particle encounters a line of block. Regions of dispersion or breakout can also be identified using the conduction velocity vector field.

### CROSS-REFERENCE TO RELATED APPLICATIONS

This application claims the benefit of U.S. provisional application No. 62/838,013, filed 24 Apr. 2019, which is hereby incorporated by reference as though fully set forth herein.

### BACKGROUND

The present disclosure relates generally to electrophysiological visualization and mapping. More specifically, the present disclosure relates to a system, method, and apparatus for generating animated electrophysiological maps for visualizing cardiac activation on the surface of a model.

Anatomical mapping, such as cardiac electrophysiological mapping, is used in numerous diagnostic and therapeutic procedures. In certain procedures, for example, various components associated with a depolarization wave are detected from electrogram signals obtained from a diagnostic catheter, and are used to generate a map, such as a local activation time (“LAT”) map, a conduction velocity (“CV”) map, a peak-to-peak (“PP”) voltage map, or a complex fractionated electrogram (“CFE”) map. Typically, such maps are static maps that employ colors and/or shading to represent parameters such as activation time, voltage, and degree of fractionation.

In some cases, it may be difficult to understand the directionality of cardiac activation wavefronts as they travel across the heart. Precise knowledge of this information is often important, however, to accurately discern patterns associated with certain cardiac arrhythmias. In some instances, such information may facilitate the detection of more complex rhythms that would otherwise be difficult to discern from more traditional, static maps.

### BRIEF SUMMARY

Disclosed herein is a method of generating an animated cardiac activation map. The method includes the steps of: receiving, at an electroanatomical mapping system, a conduction velocity map for a cardiac surface, wherein the conduction velocity map defines a conduction velocity vector field over the cardiac surface; the electroanatomical mapping system simulating flow of a particle (or, in embodiments of the disclosure, a plurality of particles) through the conduction velocity vector field; and the electroanatomical mapping system outputting an animated representation of the simulated flow of the particle (or, as applicable, the plurality of particles) through the conduction velocity vector field on a graphical representation of the cardiac surface.

In embodiments of the disclosure, the electroanatomical mapping system randomly spawns the particle within the conduction velocity vector field and may simulate flow of the particle through the conduction velocity vector field for a preset time interval.

In other embodiments of the disclosure, the electroanatomical mapping system spawns the particle within the conduction velocity vector field based upon local activation timing data for the cardiac surface (e.g., at a location within the conduction velocity vector field corresponding to a position of a cardiac activation wavefront at a current playback time of the animated cardiac activation map). The electroanatomical mapping system may simulate flow of the particle through the conduction velocity vector field for a preset time interval or, alternatively, for a time interval determined based upon the local activation timing data for the cardiac surface (e.g., until the particle either lags more than 10% of a cycle length behind the local activation timing data for the cardiac surface at an instant location of the particle or is more than 1% of the cycle length ahead of the local activation timing data for the cardiac surface at the instant location of the particle). It is also contemplated that the electroanatomical mapping may stop simulating flow of the particle through the conduction velocity vector field when the particle encounters a line of block.

The animated representation of the simulated flow of the particle through the conduction velocity vector field on the graphical representation of the cardiac surface can include a decay trail for the particle.

Also disclosed herein is a method of generating an animated cardiac activation map using an electroanatomical mapping system, including: the electroanatomical mapping system displaying a three-dimensional representation of a cardiac surface; the electroanatomical mapping system receiving a conduction velocity map for the cardiac surface, wherein the conduction velocity map defines a conduction velocity vector field over the cardiac surface; and the electroanatomical mapping system displaying a simulation of a plurality of particles flowing through the conduction velocity vector field.

According to aspects of the disclosure, the electroanatomical mapping system randomly spawns the plurality of particles within the conduction velocity vector field and displays the simulation of each particle of the plurality of particles flowing through the conduction velocity vector field for a preset time interval.

In other aspects of the disclosure, the electroanatomical mapping system spawns each particle of the plurality of particles according to a local activation timing map for the cardiac surface. The electroanatomical mapping system can then display the simulation of each particle of the plurality of particles flowing through the conduction velocity vector field for a preset time interval or, alternatively, for a time interval determined by the local activation timing map for the cardiac surface (e.g., until the particle either lags more than 10% of a cycle length behind a local activation time for the cardiac surface at an instant location of the particle or is more than 1% of the cycle length ahead of the local activation time for the cardiac surface at the instant location of the particle). It is also contemplated that the electroanatomical mapping system may stop simulating the flow of a particle when it encounters a line of block.

The instant disclosure also provides a method of graphically representing cardiac activation on a three-dimensional model of a cardiac surface using an electroanatomical mapping system. The method includes: the electroanatomical mapping system receiving a conduction velocity map for the cardiac surface, the conduction velocity map defining a conduction velocity vector field over the cardiac surface; the electroanatomical mapping system identifying a pixel within the three-dimensional model of the cardiac surface; the electroanatomical mapping system computing a dispersion metric for the identified pixel; and the electroanatomical mapping system graphically representing the pixel on the three-dimensional model of the cardiac surface as an area of dispersion when the computed dispersion metric exceeds a preset threshold.

The step of the electroanatomical mapping system computing a dispersion metric for the identified pixel can include: the electroanatomical mapping system identifying, from the conduction velocity map for the cardiac surface, a plurality of conduction velocity vectors within a preset distance of the identified pixel; for each conduction velocity vector of the plurality of conduction velocity vectors, the electroanatomical mapping system: defining a vector between the identified pixel and a location of the conduction velocity vector; and computing a dot product of the defined vector and the conduction velocity vector direction, thereby computing a plurality of dot products; and the electroanatomical mapping system summing the plurality of dot products to compute the dispersion metric for the identified pixel.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a schematic diagram of an exemplary electroanatomical mapping system.

FIG. 2 depicts an exemplary catheter that can be used in connection with aspects of the instant disclosure.

FIGS. 3A and 3B provide alphanumeric labeling conventions for electrodes carried by a multi-electrode catheter and the bipoles associated therewith.

FIG. 4 is a flowchart of representative steps that can be carried out in generating an animated activation map according to exemplary embodiments disclosed herein.

FIG. 5 illustrates a conduction velocity vector field, as output by an electroanatomical mapping system on a three-dimensional model of a cardiac geometry.

FIG. 6 illustrates a frame from an animated activation map using randomly-spawned particles according to aspects of the instant disclosure.

FIGS. 7A through 7C illustrate progressive, but not necessarily successive, frames from an animated activation map using particles spawned according to local activation timing information according to additional aspects of the instant disclosure.

FIG. 8 is an illustrative curve showing the change in opacity (that is, visibility) of a particle under flow simulation according to embodiments disclosed herein.

FIG. 9 is a flowchart of representative steps that can be carried out in generating a dispersion map according to exemplary embodiments disclosed herein.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

The present disclosure provides systems, methods, and apparatus for the creation of electrophysiology maps (e.g., electrocardiographic maps). For purposes of illustration, several exemplary embodiments will be described in detail herein with reference to cardiac electrophysiology procedures. More specifically, aspects of the disclosure will be described in the context of the creation of cardiac activation animations using electrophysiology data points collected using a high density (HD) grid catheter, such as the Advisor™ HD grid mapping catheter from Abbott Laboratories (Abbott Park, Ill.), in conjunction with an electroanatomical mapping system, such as the EnSite Precision™ cardiac mapping system, also from Abbott Laboratories. Those of ordinary skill in the art will understand, however, how to apply the teachings herein to good advantage in other contexts and/or with respect to other devices.

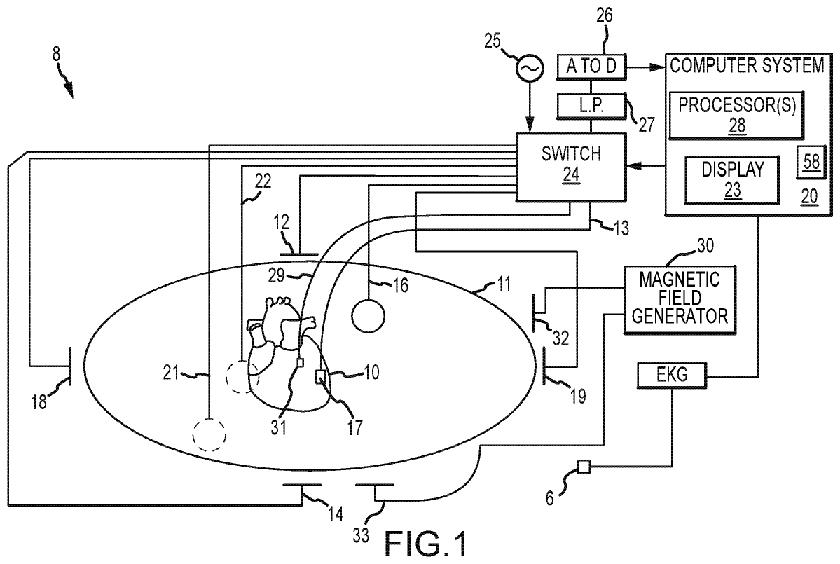

FIG. 1 shows a schematic diagram of an exemplary electroanatomical mapping system 8 for conducting cardiac electrophysiology studies by navigating a cardiac catheter and measuring electrical activity occurring in a heart 10 of a patient 11 and three-dimensionally mapping the electrical activity and/or information related to or representative of the electrical activity so measured. System 8 can be used, for example, to create an anatomical model of the patient's heart 10 using one or more electrodes. System 8 can also be used to measure electrophysiology data at a plurality of points along a cardiac surface and store the measured data in association with location information for each measurement point at which the electrophysiology data was measured, for example to create a diagnostic data map of the patient's heart 10.

As one of ordinary skill in the art will recognize, system 8 determines the location, and in some aspects the orientation, of objects, typically within a three-dimensional space, and expresses those locations as position information determined relative to at least one reference.

For simplicity of illustration, the patient 11 is depicted schematically as an oval. In the embodiment shown in FIG. 1, three sets of surface electrodes (e.g., patch electrodes) are shown applied to a surface of the patient 11, defining three generally orthogonal axes, referred to herein as an x-axis, a y-axis, and a z-axis. In other embodiments the electrodes could be positioned in other arrangements, for example multiple electrodes on a particular body surface. As a further alternative, the electrodes do not need to be on the body surface, but could be positioned internally to the body.

In FIG. 1, the x-axis surface electrodes 12, 14 are applied to the patient along a first axis, such as on the lateral sides of the thorax region of the patient (e.g., applied to the patient's skin underneath each arm) and may be referred to as the Left and Right electrodes. The y-axis electrodes 18, 19 are applied to the patient along a second axis generally orthogonal to the x-axis, such as along the inner thigh and neck regions of the patient, and may be referred to as the Left Leg and Neck electrodes. The z-axis electrodes 16, 22 are applied along a third axis generally orthogonal to both the x-axis and the y-axis, such as along the sternum and spine of the patient in the thorax region, and may be referred to as the Chest and Back electrodes. The heart 10 lies between these pairs of surface electrodes 12/14, 18/19, and 16/22.

An additional surface reference electrode (e.g., a “belly patch”) 21 provides a reference and/or ground electrode for the system 8. The belly patch electrode 21 may be an alternative to a fixed intra-cardiac electrode 31, described in further detail below. It should also be appreciated that, in addition, the patient 11 may have most or all of the conventional electrocardiogram (“ECG” or “EKG”) system leads in place. In certain embodiments, for example, a standard set of 12 ECG leads may be utilized for sensing electrocardiograms on the patient's heart 10. This ECG information is available to the system 8 (e.g., it can be provided as input to computer system 20). Insofar as ECG leads are well understood, and for the sake of clarity in the figures, only a single lead 6 and its connection to computer 20 is illustrated in FIG. 1.

A representative catheter 13 having at least one electrode 17 is also shown. This representative catheter electrode 17 is referred to as the “roving electrode,” “moving electrode,” or “measurement electrode” throughout the specification. Typically, multiple electrodes 17 on catheter 13, or on multiple such catheters, will be used. In one embodiment, for example, the system 8 may comprise sixty-four electrodes on twelve catheters disposed within the heart and/or vasculature of the patient. In other embodiments, system 8 may utilize a single catheter that includes multiple (e.g., eight) splines, each of which in turn includes multiple (e.g., eight) electrodes.

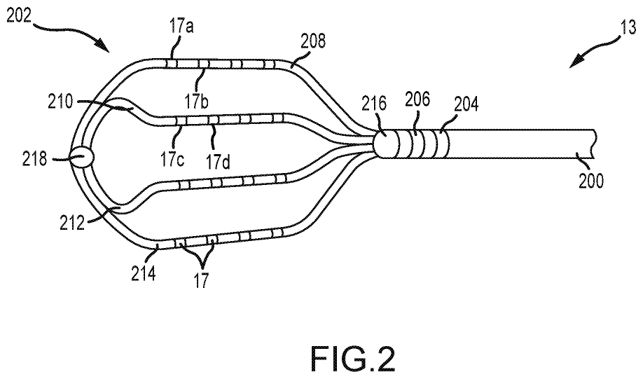

The foregoing embodiments are merely exemplary, however, and any number of electrodes and/or catheters may be used. For example, for purposes of this disclosure, a segment of an exemplary multi-electrode catheter, and in particular an HD grid catheter, is shown in FIG. 2. HD grid catheter 13 includes a catheter body 200 coupled to a paddle 202. Catheter body 200 can further include first and second body electrodes 204, 206, respectively. Paddle 202 can include a first spline 208, a second spline 210, a third spline 212, and a fourth spline 214, which are coupled to catheter body 200 by a proximal coupler 216 and to each other by a distal coupler 218. In one embodiment, first spline 208 and fourth spline 214 can be one continuous segment and second spline 210 and third spline 212 can be another continuous segment. In other embodiments, the various splines 208, 210, 212, 214 can be separate segments coupled to each other (e.g., by proximal and distal couplers 216, 218, respectively). It should be understood that HD catheter 13 can include any number of splines; the four-spline arrangement shown in FIG. 2 is merely exemplary.

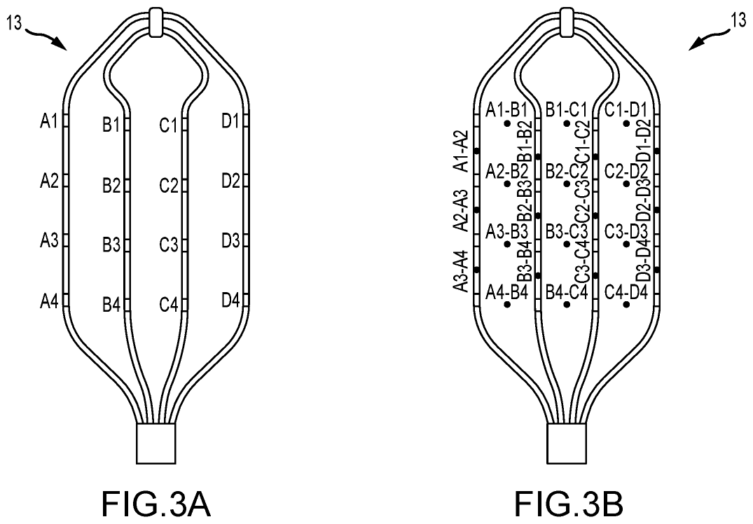

As described above, splines 208, 210, 212, 214 can include any number of electrodes 17; in FIG. 2, sixteen electrodes 17 are shown arranged in a four-by-four array. It should also be understood that electrodes 17 can be evenly and/or unevenly spaced, as measured both along and between splines 208, 210, 212, 214. For purposes of easy reference in this description, FIG. 3A provides alphanumeric labels for electrodes 17.

As those of ordinary skill in the art will recognize, any two neighboring electrodes 17 define a bipole. Thus, the 16 electrodes 17 on catheter 13 define a total of 42 bipoles —12 along splines (e.g., between electrodes 17a and 17b, or between electrodes 17c and 17d), 12 across splines (e.g., between electrodes 17a and 17c, or between electrodes 17b and 17d), and 18 diagonally between splines (e.g., between electrodes 17a and 17d, or between electrodes 17b and 17c).

For ease of reference in this description, FIG. 3B provides alphanumeric labels for the along- and across-spline bipoles. FIG. 3B omits alphanumeric labels for the diagonal bipoles, but this is only for the sake of clarity in the illustration. It is expressly contemplated that the teachings herein can also be applied with respect to the diagonal bipoles.

Any bipole can, in turn, be used to generate a bipolar electrogram according to techniques that will be familiar to those of ordinary skill in the art. Moreover, these bipolar electrograms can be combined (e.g., linearly combined) to generate electrograms, again including activation timing information, in any direction of the plane of catheter 13 by computing an E-field loop for a clique of electrodes. U.S. application Ser. No. 15/953,155, which is hereby incorporated by reference as though fully set forth herein, discloses details of computing an E-field loop for a clique of electrodes on a HD grid catheter.

In any event, catheter 13 can be used to simultaneously collect a plurality of electrophysiology data points for the various bipoles defined by electrodes 17 thereon, with each such electrophysiology data point including both localization information (e.g., position and orientation of a selected bipole) and an electrogram signal for the selected bipole. For purposes of illustration, methods according to the instant disclosure will be described with reference to individual electrophysiology data points collected by catheter 13. It should be understood, however, that the teachings herein can be applied, in serial and/or in parallel, to multiple electrophysiology data points collected by catheter 13.

Catheter 13 (or multiple such catheters) are typically introduced into the heart and/or vasculature of the patient via one or more introducers and using familiar procedures. Indeed, various approaches to introduce catheter 13 into a patient's heart, such as transseptal approaches, will be familiar to those of ordinary skill in the art, and therefore need not be further described herein.

Since each electrode 17 lies within the patient, location data may be collected simultaneously for each electrode 17 by system 8. Similarly, each electrode 17 can be used to gather electrophysiological data from the cardiac surface (e.g., surface electrograms). The ordinarily skilled artisan will be familiar with various modalities for the acquisition and processing of electrophysiology data points (including, for example, both contact and non-contact electrophysiological mapping), such that further discussion thereof is not necessary to the understanding of the techniques disclosed herein. Likewise, various techniques familiar in the art can be used to generate a graphical representation of a cardiac geometry and/or of cardiac electrical activity from the plurality of electrophysiology data points. Moreover, insofar as the ordinarily skilled artisan will appreciate how to create electrophysiology maps from electrophysiology data points, the aspects thereof will only be described herein to the extent necessary to understand the present disclosure.

Returning now to FIG. 1, in some embodiments, an optional fixed reference electrode 31 (e.g., attached to a wall of the heart 10) is shown on a second catheter 29. For calibration purposes, this electrode 31 may be stationary (e.g., attached to or near the wall of the heart) or disposed in a fixed spatial relationship with the roving electrodes (e.g., electrodes 17), and thus may be referred to as a “navigational reference” or “local reference.” The fixed reference electrode 31 may be used in addition or alternatively to the surface reference electrode 21 described above. In many instances, a coronary sinus electrode or other fixed electrode in the heart 10 can be used as a reference for measuring voltages and displacements; that is, as described below, fixed reference electrode 31 may define the origin of a coordinate system.

Each surface electrode is coupled to a multiplex switch 24, and the pairs of surface electrodes are selected by software running on a computer 20, which couples the surface electrodes to a signal generator 25. Alternately, switch 24 may be eliminated and multiple (e.g., three) instances of signal generator 25 may be provided, one for each measurement axis (that is, each surface electrode pairing).

The computer 20 may comprise, for example, a conventional general-purpose computer, a special-purpose computer, a distributed computer, or any other type of computer. The computer 20 may comprise one or more processors 28, such as a single central processing unit (“CPU”), or a plurality of processing units, commonly referred to as a parallel processing environment, which may execute instructions to practice the various aspects described herein.

Generally, three nominally orthogonal electric fields are generated by a series of driven and sensed electric dipoles (e.g., surface electrode pairs 12/14, 18/19, and 16/22) in order to realize catheter navigation in a biological conductor. Alternatively, these orthogonal fields can be decomposed and any pairs of surface electrodes can be driven as dipoles to provide effective electrode triangulation. Likewise, the electrodes 12, 14, 18, 19, 16, and 22 (or any number of electrodes) could be positioned in any other effective arrangement for driving a current to or sensing a current from an electrode in the heart. For example, multiple electrodes could be placed on the back, sides, and/or belly of patient 11. Additionally, such non-orthogonal methodologies add to the flexibility of the system. For any desired axis, the potentials measured across the roving electrodes resulting from a predetermined set of drive (source-sink) configurations may be combined algebraically to yield the same effective potential as would be obtained by simply driving a uniform current along the orthogonal axes.

Thus, any two of the surface electrodes 12, 14, 16, 18, 19, 22 may be selected as a dipole source and drain with respect to a ground reference, such as belly patch 21, while the unexcited electrodes measure voltage with respect to the ground reference. The roving electrodes 17 placed in the heart 10 are exposed to the field from a current pulse and are measured with respect to ground, such as belly patch 21. In practice the catheters within the heart 10 may contain more or fewer electrodes than the sixteen shown, and each electrode potential may be measured. As previously noted, at least one electrode may be fixed to the interior surface of the heart to form a fixed reference electrode 31, which is also measured with respect to ground, such as belly patch 21, and which may be defined as the origin of the coordinate system relative to which system 8 measures positions. Data sets from each of the surface electrodes, the internal electrodes, and the virtual electrodes may all be used to determine the location of the roving electrodes 17 within heart 10.

The measured voltages may be used by system 8 to determine the location in three-dimensional space of the electrodes inside the heart, such as roving electrodes 17 relative to a reference location, such as reference electrode 31. That is, the voltages measured at reference electrode 31 may be used to define the origin of a coordinate system, while the voltages measured at roving electrodes 17 may be used to express the location of roving electrodes 17 relative to the origin. In some embodiments, the coordinate system is a three-dimensional (x, y, z) Cartesian coordinate system, although other coordinate systems, such as polar, spherical, and cylindrical coordinate systems, are contemplated.

As should be clear from the foregoing discussion, the data used to determine the location of the electrode(s) within the heart is measured while the surface electrode pairs impress an electric field on the heart. The electrode data may also be used to create a respiration compensation value used to improve the raw location data for the electrode locations as described, for example, in U.S. Pat. No. 7,263,397, which is hereby incorporated herein by reference in its entirety. The electrode data may also be used to compensate for changes in the impedance of the body of the patient as described, for example, in U.S. Pat. No. 7,885,707, which is also incorporated herein by reference in its entirety.

Therefore, in one representative embodiment, system 8 first selects a set of surface electrodes and then drives them with current pulses. While the current pulses are being delivered, electrical activity, such as the voltages measured with at least one of the remaining surface electrodes and in vivo electrodes, is measured and stored. Compensation for artifacts, such as respiration and/or impedance shifting, may be performed as indicated above.

In aspects of the disclosure, system 8 can be a hybrid system that incorporates both impedance-based (e.g., as described above) and magnetic-based localization capabilities. Thus, for example, system 8 can also include a magnetic source 30, which is coupled to one or more magnetic field generators. In the interest of clarity, only two magnetic field generators 32 and 33 are depicted in FIG. 1, but it should be understood that additional magnetic field generators (e.g., a total of six magnetic field generators, defining three generally orthogonal axes analogous to those defined by patch electrodes 12, 14, 16, 18, 19, and 22) can be used without departing from the scope of the present teachings. Likewise, those of ordinary skill in the art will appreciate that, for purposes of localizing catheter 13 within the magnetic fields so generated, can include one or more magnetic localization sensors (e.g., coils).

In some embodiments, system 8 is the EnSite™ Velocity™ or EnSite Precision™ cardiac mapping and visualization system of Abbott Laboratories. Other localization systems, however, may be used in connection with the present teachings, including for example the RHYTHMIA HDX™ mapping system of Boston Scientific Corporation (Marlborough, Mass.), the CARTO navigation and location system of Biosense Webster, Inc. (Irvine, Calif.), the AURORA® system of Northern Digital Inc. (Waterloo, Ontario), Sterotaxis, Inc.'s NIOBE® Magnetic Navigation System (St. Louis, Mo.), as well as MediGuide™ Technology from Abbott Laboratories.

The localization and mapping systems described in the following patents (all of which are hereby incorporated by reference in their entireties) can also be used with the present invention: U.S. Pat. Nos. 6,990,370; 6,978,168; 6,947,785; 6,939,309; 6,728,562; 6,640,119; 5,983,126; and 5,697,377.

Aspects of the disclosure relate to electrophysiological mapping, and in particular to generating animated maps of cardiac activation. Graphical representations of such animated maps can be output, for example on display 23. System 8 can therefore include an activation animation module 58 that can be used to generate an animated cardiac activation map and output the same (e.g., on display 23).

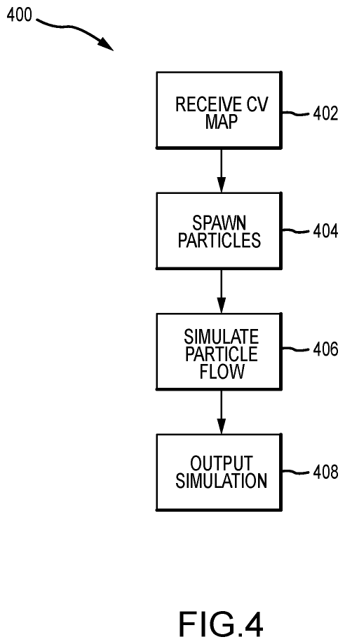

One exemplary method according to the present teachings will be explained with reference to the flowchart 400 of representative steps presented as FIG. 4. In some embodiments, for example, flowchart 400 may represent several exemplary steps that can be carried out by electroanatomical mapping system 8 of FIG. 1 (e.g., by processor 28 and/or activation animation module 58). It should be understood that the representative steps described below can be either hardware- or software-implemented. For the sake of explanation, the term “signal processor” may be used herein to describe both hardware- and software-based implementations of the teachings herein.

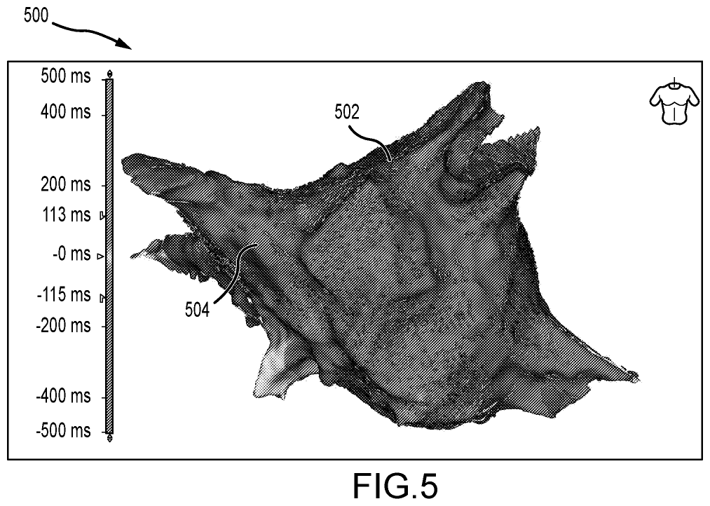

In block 402, system 8 receives a conduction velocity map for a cardiac surface. According to aspects of the disclosure, the conduction velocity map defines a conduction velocity vector field over the cardiac surface. For instance, FIG. 5 graphically depicts a conduction velocity map 500 using a plurality of arrows 502, each representing the conduction velocity vector at a particular location, on a three-dimensional model of a cardiac geometry 504.

Insofar as conduction velocity maps are generally known to those of ordinary skill in the art, they need not be described in detail herein. For purposes of illustration, however, United States patent application publication no. 2017/0042449, which is hereby incorporated by reference as though fully set forth herein, discloses the computation of conduction velocity vectors using HD grid catheter 13 and the graphical representation of such conduction velocity vectors on a cardiac surface mode (e.g., as arrows).

In block 404, system 8 spawns particles within the conduction velocity vector field defined by the conduction velocity map. In some embodiments of the disclosure, system 8 randomly spawns particles within the conduction velocity vector field defined by the conduction velocity map.

In other embodiments of the disclosure, system 8 spawns particles within the conduction velocity vector field based upon a local activation timing map for the cardiac surface. Like conduction velocity maps, local activation timing maps, which describe the timing of cardiac activation relative to a temporal datum (e.g., a time to), will be familiar to those of ordinary skill in the art and need not be further described herein.

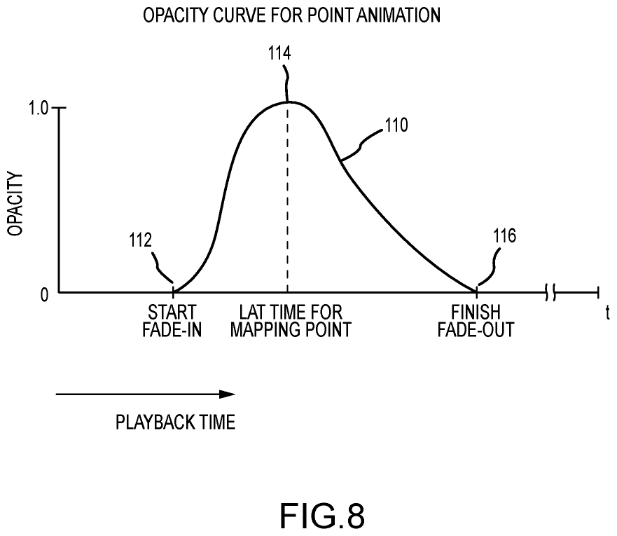

In particular, and as described in further detail below in connection with FIG. 8, it is contemplated that, for any given location on the cardiac surface, system 8 can spawn a particle at that location when the local activation time at that location coincides with a playback time of the animated cardiac activation map. Put another way, insofar as a local activation timing map reflects the position of a cardiac activation wavefront at any given point in time, relative to the temporal datum, system 8 spawns particles at locations as a cardiac activation wavefront approaches those locations.

In block 406, system 8 simulates the flow of the spawned particles through the conduction velocity vector field. The simulation timestep can be chosen such that, when the simulation is output in block 408 (described below), a spawned particle does not skip pixels (e.g., the spawned particle appears to flow smoothly by moving from one pixel to an adjacent pixel). Likewise, the simulation timestep should be granular enough to avoid a spawned particle appearing to jump across a line of block when output in block 408.

Thus, in aspects of the disclosure, the simulation timestep can be calculated using the current conduction velocity and the pixel resolution such that, frame-to-frame, a spawned particle travels at most one pixel. For instance, if the pixel resolution is 0.33 mm/pixel, and the current conduction velocity is 0.5 mm/ms, then the simulation timestep can be calculated as (0.33 mm/pixel)/(0.55 mm/ms)=0.167 ms/pixel, or a simulation timestep of 0.167 ms.

Those of ordinary skill in the art will be familiar with simulations of particle flow through a vector field, such that this aspect of the disclosure need not be described in extensive detail herein. It should be understood, however, that the conduction velocity vector field is three-dimensional, but that the particle flow is simulated over a surface in block 408. In embodiments of the disclosure, therefore, particles are first spawned on the surface, and then iteratively moved in three-dimensions according to the conduction velocity vector field (e.g., as influenced by the local conduction velocity vector at the instant location of the spawned particle over the selected simulation timestep) and projected back to the surface.

According to aspects of the disclosure, system 8 simulates the flow of a spawned particle through the conduction velocity vector field for a “simulation time,” which, in embodiments of the disclosure, can be a preset time interval. The use of a preset simulation time may be particularly desirable for randomly-spawned particles, though preset simulation times may also be used for particles spawned according to local activation time data. In embodiments of the disclosure, the simulation time will be at least about one half of a cycle length.

Those of ordinary skill in the art will also appreciate that there may be a direct relationship between the simulation time and the playback speed of the simulation as output in block 408. That is, as the playback speed of the simulation as output in block 408 increases, the length of the time interval over which system 8 simulates the flow of a spawned particle may also increase.

In other aspects of the disclosure, the simulation time is determined based upon the local activation timing map for the cardiac surface. In other words, in embodiments of the disclosure, particles may both spawn and die based upon local timing information. For instance, in aspects of the disclosure, a particle may die if it falls behind the activation wavefront by more than about 10% of the cycle length and/or if it gets ahead of the activation wavefront by more than about 1% of the cycle length, with typical cycle lengths being between about 220 ms and about 1000 ms.

In still other aspects of the disclosure, system 8 simulates the flow of a spawned particle through the conduction velocity vector field until it encounters a line of block, at which point the particle dies. U.S. provisional application No. 62/835,937, filed 18 Apr. 2019, which is hereby incorporated by reference as though fully set forth herein, discloses methods, apparatus, and systems for identifying lines of block using electrophysiology maps (and, in particular, local activation timing and/or conduction velocity maps as also described herein).

In block 408, system 8 outputs an animated representation of the simulated particle flow on a graphical representation of the cardiac surface (e.g., on display 23). This output is referred to herein as an animated cardiac activation map. It should be understood that the amount of time over which a simulation is output as an animated representation (referred to herein as the “display time”) can be much longer than the simulation time in order to make the particle flow visually apparent to a practitioner. For instance, although the simulation time may be about a half cycle length (e.g., a small fraction of a second), the animated representation of the simulation may be slowed down so as to have a display time of about one second.

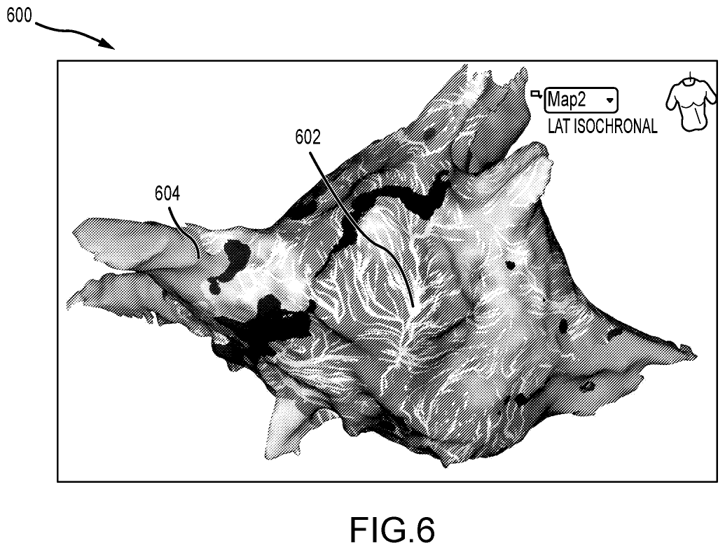

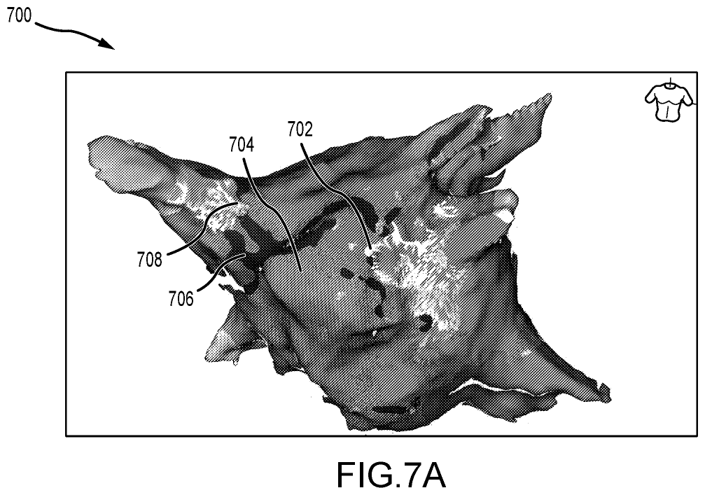

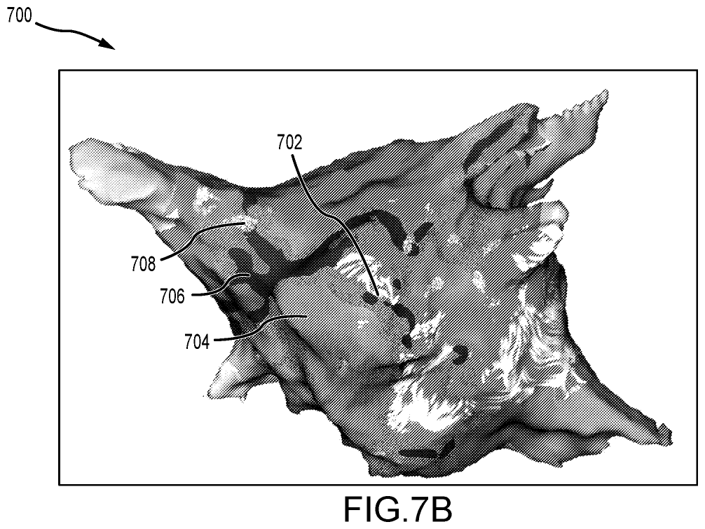

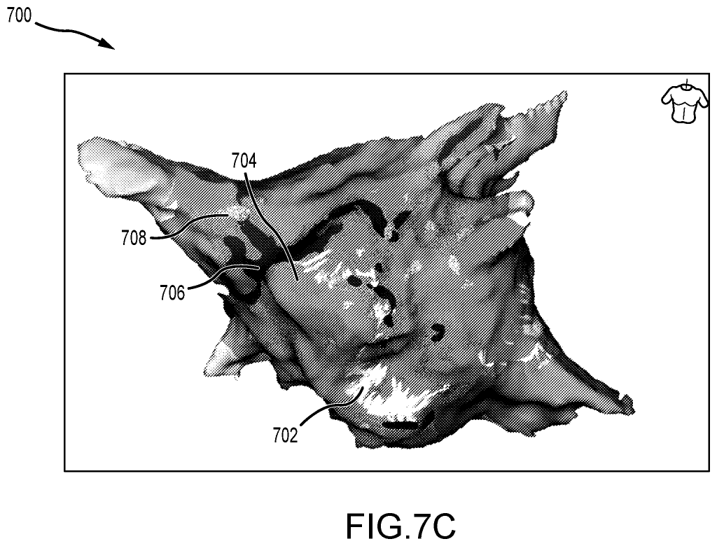

FIGS. 6 and 7A through 7C depict still frames from animated cardiac activation maps according to various aspects of the instant disclosure. For instance, FIG. 6 depicts a single still frame from an animated cardiac activation map 600 that reflects the flow of randomly generated particles as flow lines 602 on cardiac geometry 604. By observing flow lines 602 animating over cardiac geometry 604, a practitioner can visually identify potential ablation targets (e.g., areas of dispersion or breakout).

FIGS. 7A-7C, on the other hand, depict three progressive (but not necessarily successive) frames of an animated cardiac activation map 700, where system 8 spawns particles 702 according to local activation timing information, simulates particles 702 over cardiac geometry 704, with particles 702 dying when they encounter a line of block (e.g., line 706). By observing particles 702 as they move over cardiac geometry 704 in successive frames, a practitioner can visually recognize propagation of the cardiac activation wavefront.

FIG. 8 is a representative curve for the visibility (opacity) of any given location on the cardiac geometry of a particle under simulation according to aspects of the instant disclosure. Particle spawn at the given location begins at time 112, with the particle fading in (e.g., as the activation wavefront approaches the particular location). Particle spawn is complete at time 114 (e.g., the time at which the activation wavefront coincides with the given location), as the particle has its maximum visibility at the given location. The presence of the particle at the given location then fades out along slope 110 (e.g., as the activation wavefront moves away from the given location), until disappearing completely at time 116. This graphical approach gives the impression that particles leave behind gradually-disappearing trails (referred to herein as “decay trails”) as they flow over the cardiac surface.

The animated cardiac activation maps disclosed herein can be displayed not just as standalone maps, but also in conjunction with other electrophysiology maps (e.g., rendered as an additional layer upon a peak-to-peak voltage map, a complex fractionated electrogram map, a LAT map, and/or the like). For instance, in embodiments of the disclosure, an animated cardiac activation map can be displayed in conjunction with a dispersion map to aid a practitioner in visually identifying regions of dispersion or breakout on the cardiac surface.

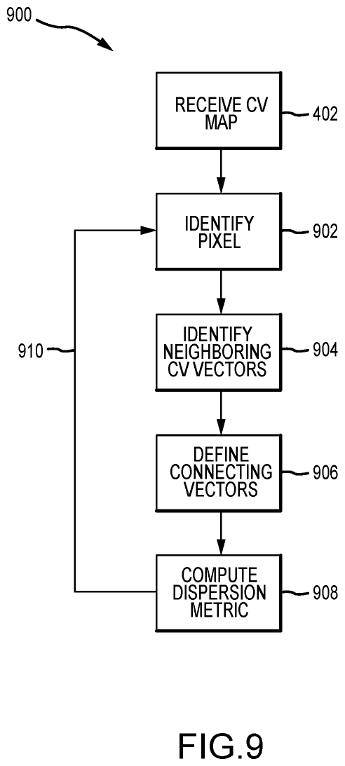

FIG. 9 represents a flowchart 900 of representative steps corresponding to an exemplary method of computing a dispersion map according to the present teachings. In some embodiments, flowchart 900 may represent several exemplary steps that can be carried out by electroanatomical mapping system 8 of FIG. 1 (e.g., by processor 28 and/or activation animation module 58), though it should be understood that the representative steps described below can be either hardware- or software-implemented. For the sake of explanation, the term “signal processor” may be used herein to describe both hardware- and software-based implementations of the teachings herein.

Flowchart 900 begins with system 8 receiving a conduction velocity map in block 402. Block 402 is described above; in the interest of brevity, details of block 402 will not be repeated here.

In block 902, system 8 identifies a pixel within a model of the cardiac surface (e.g., model 504 of FIG. 5, model 604 of FIG. 6, or model 704 of FIGS. 7A-7C).

In block 904, system 8 identifies, from the conduction velocity map received in block 402, a plurality of conduction velocity vectors within a preset distance of the pixel identified in block 902. These conduction velocity vectors are referred to herein as “neighboring conduction velocity vectors.” In embodiments of the disclosure, the preset distance can be between about 0 mm and about 30 mm. Particular embodiments of the disclosure utilize preset distances of about 2 mm or about 7 mm.

It should be understood that the neighboring conduction velocity vectors may include not only conduction velocity vectors for electrophysiology data points directly measured by electrodes 17 on catheter 13, but also for other locations on the cardiac surface model (e.g., other pixels on the cardiac geometry). These additional locations may be assigned values for relevant electrophysiology metrics (e.g., conduction velocity, local activation time, and the like) via interpolation from electrophysiology data points directly measured by electrodes 17 on catheter 13. More generally, it is contemplated that the teachings herein can be applied not only to electrophysiology data points directly measured by electrodes 17 on catheter 13, but also to individual pixels within the cardiac surface model.

In block 906, system 8 defines vectors, referred to herein as “connecting vectors,” between the pixel identified in block 902 and each neighboring conduction velocity vector.

In block 908, system 8 computes a dispersion metric, such as by using the sum and/or mean of the dot products between the neighboring conduction velocity vectors and their respective connecting vectors. Thus, in embodiments of the disclosure, system 8 normalizes the neighboring conduction velocity vectors and their respective connecting vectors into unit vectors, computes the dot products therebetween, and then computes the sum and/or mean of the dot products.

Regions of parallel or homogenous conduction will have dispersion metrics (e.g., mean dot products) close to zero. Large, positive dispersion metrics suggest that the identified pixel is an area of dispersion or breakout. On the other hand, large, negative dispersion metrics suggest that the identified pixel is an area of collision.

By computing dispersion metrics for multiple locations, as shown by loop 910 (e.g., for all pixels in the cardiac geometry), system 8 can compute a dispersion map. System 8 can, in turn, graphically represent the dispersion map (e.g., as stippled regions 708 in FIGS. 7A-7C).

Indeed, it is contemplated that the thresholds at which a dispersion metric is considered an indicator of breakout (or collision) can be user defined. For instance, a suitable control (e.g., a slider) can be provided on a graphical user interface on display 23; by adjusting the slider, a practitioner can adjust the threshold until regions of breakout (or collision) appear on the dispersion map.

Although several embodiments have been described above with a certain degree of particularity, those skilled in the art could make numerous alterations to the disclosed embodiments without departing from the spirit or scope of this invention.

For example, the teachings herein can be applied in real time (e.g., during an electrophysiology study) or during post-processing (e.g., to electrophysiology data points collected during an electrophysiology study performed at an earlier time).

Another example is the following alternative dispersion metric to the dispersion metric described above in connection with FIG. 9. In particular, once the neighboring conduction vectors are normalized to unit vectors, their mean (referred to herein as “R”) can be computed; the value of R will be between zero and one. In areas of parallel or homogenous conduction, R will be close to one. In areas of breakout/dispersion or collision, R will be closer to zero. Variations are also contemplated; for instance, because one might intuitively associate increasing dispersion metric values with increasing dispersion, the reciprocal of R or the square root of the natural logarithm of R could be used instead.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A method of generating an animated cardiac activation map, the method comprising:
receiving, at an electroanatomical mapping system, a conduction velocity map for a cardiac surface, wherein the conduction velocity map defines a conduction velocity vector field over the cardiac surface;
the electroanatomical mapping system simulating flow of a particle through the conduction velocity vector field; and
the electroanatomical mapping system outputting an animated representation of the simulated flow of the particle through the conduction velocity vector field on a graphical representation of the cardiac surface.

2. The method according to claim 1, wherein the electroanatomical mapping system simulating flow of a particle through the conduction velocity vector field comprises the electroanatomical mapping system randomly spawning the particle within the conduction velocity vector field.

3. The method according to claim 2, wherein the electroanatomical mapping system simulating flow of the particle through the conduction velocity vector field further comprises the electroanatomical mapping system simulating flow of the particle through the conduction velocity vector field for a preset time interval.

4. The method according to claim 1, wherein the electroanatomical mapping system simulating flow of a particle through the conduction velocity vector field comprises the electroanatomical mapping system spawning the particle within the conduction velocity vector field based upon local activation timing data for the cardiac surface.

5. The method according to claim 4, wherein the electroanatomical mapping system spawning the particle within the conduction velocity vector field based upon local activation timing data for the cardiac surface further comprises the electroanatomical mapping system spawning the particle at a location within the conduction velocity vector field corresponding to a position of a cardiac activation wavefront at a current playback time of the animated cardiac activation map.

6. The method according to claim 4, wherein the electroanatomical mapping system simulating flow of the particle through the conduction velocity vector field further comprises the electroanatomical mapping system simulating flow of the particle through the conduction velocity vector field for a preset time interval.

7. The method according to claim 4, wherein the electroanatomical mapping system simulating flow of the particle through the conduction velocity vector field further comprises the electroanatomical mapping system simulating flow of the particle through the conduction velocity vector field for a time interval determined based upon the local activation timing data for the cardiac surface.

8. The method according to claim 7, wherein the time interval ends when the particle either lags more than 10% of a cycle length behind the local activation timing data for the cardiac surface at an instant location of the particle or is more than 1% of the cycle length ahead of the local activation timing data for the cardiac surface at the instant location of the particle.

9. The method according to claim 4, wherein the electroanatomical mapping system simulating flow of the particle through the conduction velocity vector field further comprises the electroanatomical mapping system simulating flow of the particle through the conduction velocity vector field until the particle encounters a line of block.

10. The method according to claim 1, wherein:
the electroanatomical mapping system simulating flow of a particle through the conduction velocity vector field comprises the electroanatomical mapping system simulating flow of a plurality of particles through the conduction velocity vector field, and
the electroanatomical mapping system outputting an animated representation of the simulated flow of the particle through the conduction velocity vector field on a graphical representation of the cardiac surface comprises the electroanatomical mapping system outputting an animated representation of the simulated flow of the plurality of particles through the conduction velocity vector field on the graphical representation of the cardiac surface.

11. The method according to claim 1, wherein the animated representation of the simulated flow of the particle through the conduction velocity vector field on the graphical representation of the cardiac surface further comprises a decay trail for the particle.

12. A method of generating an animated cardiac activation map using an electroanatomical mapping system, the method comprising:
the electroanatomical mapping system displaying a three-dimensional representation of a cardiac surface;
the electroanatomical mapping system receiving a conduction velocity map for the cardiac surface, wherein the conduction velocity map defines a conduction velocity vector field over the cardiac surface; and
the electroanatomical mapping system displaying a simulation of a plurality of particles flowing through the conduction velocity vector field.

13. The method according to claim 12, wherein the electroanatomical mapping system displaying a simulation of a plurality of particles flowing through the conduction velocity vector field comprises:
the electroanatomical mapping system randomly spawning the plurality of particles within the conduction velocity vector field; and
the electroanatomical mapping system displaying the simulation of each particle of the plurality of particles flowing through the conduction velocity vector field for a preset time interval.

14. The method according to claim 12, wherein the electroanatomical mapping system displaying a simulation of a plurality of particles flowing through the conduction velocity vector field comprises the electroanatomical mapping system spawning each particle of the plurality of particles according to a local activation timing map for the cardiac surface.

15. The method according to claim 14, wherein the electroanatomical mapping system displaying a simulation of a plurality of particles flowing through the conduction velocity vector field further comprises the electroanatomical mapping system displaying the simulation of each particle of the plurality of particles flowing through the conduction velocity vector field for a preset time interval.

16. The method according to claim 14, wherein the electroanatomical mapping system displaying a simulation of a plurality of particles flowing through the conduction velocity vector field further comprises the electroanatomical mapping system displaying the simulation of each particle of the plurality of particles flowing through the conduction velocity vector field for a time interval determined by the local activation timing map for the cardiac surface.

17. The method according to claim 16, wherein the time interval ends when the particle either lags more than 10% of a cycle length behind a local activation time for the cardiac surface at an instant location of the particle or is more than 1% of the cycle length ahead of the local activation time for the cardiac surface at the instant location of the particle.

18. The method according to claim 14, wherein the electroanatomical mapping system displaying a simulation of a plurality of particles flowing through the conduction velocity vector field further comprises the electroanatomical mapping system displaying the simulation of each particle of the plurality of particles flowing through the conduction velocity vector field until the respective particle encounters a line of block.

19. A method of graphically representing cardiac activation on a three-dimensional model of a cardiac surface using an electroanatomical mapping system, the method comprising:
the electroanatomical mapping system receiving a conduction velocity map for the cardiac surface, the conduction velocity map defining a conduction velocity vector field over the cardiac surface;
the electroanatomical mapping system identifying a pixel within the three-dimensional model of the cardiac surface;
the electroanatomical mapping system computing a dispersion metric for the identified pixel; and
the electroanatomical mapping system graphically representing the pixel on the three-dimensional model of the cardiac surface as an area of dispersion when the computed dispersion metric exceeds a preset threshold.

20. The method according to claim 19, wherein the electroanatomical mapping system computing a dispersion metric for the identified pixel comprises:
the electroanatomical mapping system identifying, from the conduction velocity map for the cardiac surface, a plurality of conduction velocity vectors within a preset distance of the identified pixel;
for each conduction velocity vector of the plurality of conduction velocity vectors, the electroanatomical mapping system:
defining a vector between the identified pixel and a location of the conduction velocity vector; and
computing a dot product of the defined vector and the conduction velocity vector direction,
thereby computing a plurality of dot products; and
the electroanatomical mapping system summing the plurality of dot products to compute the dispersion metric for the identified pixel.
